(19) United States
(12) Patent Application Publication
(10) Pub. No.: US 2025/0378479 A1
(43) Pub. Date: Dec. 11, 2025

(54) MONITORING DEVICE, NETWORK NODE AND METHODS FOR HANDLING DATA

(71) Applicant: Telefonaktiebolaget LM Ericsson (publ), Stockholm (SE)

(72) Inventors: Jalil TAGHIA, Stockholm (SE); Selim ICKIN, Danderyd (SE); Carmen LEE ALTMANN, Täby (SE); Konstantinos VANDIKAS, Solna (SE)

(21) Appl. No.: 18/876,963

(22) PCT Filed: Sep. 13, 2022
(86) PCT No.: PCT/EP2022/075396

§ 371 Date: Dec. 19, 2024

(30) Foreign Application Priority Data
Jul. 1, 2022 (GR) 20220100527

(51) Int. Cl. G06Q 30/0601; A61B 5/16

(52) U.S. Cl. CPC G06Q 30/0631; A61B 5/165

(57) ABSTRACT

A monitoring device for handling brain activity data of a user. The monitoring device obtains sensor data from a plurality of sensors indicating a brain activity of one or more regions in a brain of the user. The monitoring device constructs a similarity module of brain activities based on an activity configuration defining one or more of the sensors to gather sensor data from and obtained sensor data from the one or more sensors, wherein the similarity module includes one or more groups of sensor data related to one another. The monitoring device performs an interaction analysis within and/or between the one or more groups in the similarity module by creating a network of interactions within and/or between the one or more groups. The monitoring device creates an aggregated activity graph based on the constructed similarity module, the performed interaction analysis, and a configured aggregation level of sensor data.

### TECHNICAL FIELD

Embodiments herein relate to a monitoring device, a network node, and methods performed therein for handling data. Furthermore, a computer program product and a computer readable storage medium are also provided herein. In particular, embodiments herein relate to handling or processing brain activity data of a user.

### BACKGROUND

Consider an immersive scenario involving multiple senses as shown in FIG. 1. Seen mind as a unifying sense, monitoring brain activities of a user during the immersive scenario can provide useful information about how the content is perceived by the user-for example whether the user enjoyed the experience and if so, which senses contributed mostly. Such information can be used by the content provider for generation or recommendation of personalized contents for the user, for example, during a gaming session the user may be recommended personalized content based on enjoyed experience. FIG. 1 shows an immersive session that may involve a number of senses. Mind can be seen as a unifying entity.

Recommendation systems based on a user's brain activity have been previously studied in for example, Beheshti, A., Yakhchi, S., Mousaeirad, S., Ghafari, S. M., Goluguri, S. R., & Edrisi, M. A. (2020). Towards Cognitive Recommender Systems. Algorithms, 13, 176 and US20120072289A1. One outstanding challenge is privacy as readings from brain activities are highly privacy sensitive. Directly sharing such information with content generators may not be possible. It might be possible to secure such information, but it can become prohibitively costly for certain use-cases due to their inherently large size.

### SUMMARY

As part of developing embodiments herein one or more problems have been identified. Current solutions are based on readings from a brain that are not privacy-preserving by construction. Securing such information may become costly in terms of volume and complexity and the employed method of security can itself be subject to leakage of information and specialized attacks.

Current solutions are focused on security meaning that they create a secure environment protected by Public/Private Key infrastructure to collect readings from the brain. This is not privacy-preserving process since a trusted process running in such an environment can identify individuals. Techniques from the area of Multi-party computation may be applied to such a problem and enable privacy but they are very costly computationally speaking in relation to the number of participants which makes them practically unusable when dealing with large numbers of users.

An object of embodiments herein is to provide an efficient and privacy-preserving way of handling data in a communication network.

According to an aspect the object may be achieved by a method performed by a monitoring device for handling brain activity data of a user. The monitoring device obtains sensor data from a plurality of sensors, wherein the sensor data indicates a brain activity of one or more regions in a brain of the user. The monitoring device constructs a similarity module of brain activities based on an activity configuration defining one or more sensors out of the plurality of sensors to gather sensor data from, and obtained sensor data from the one or more sensors, wherein the similarity module comprises one or more groups of sensor data related to one another. The monitoring device performs an interaction analysis within and/or between the one or more groups in the similarity module by creating a network of interactions within and/or between the one or more groups. The monitoring device creates an aggregated activity graph based on the constructed similarity module, the performed interaction analysis, and a configured aggregation level of sensor data; and provides the aggregated activity graph to a network node.

According to another aspect the object may be achieved by a method performed by a network node for handling brain activity data of a user. The network node receives from a monitoring device related to the user, an aggregated activity graph, wherein the aggregated activity graph is based on a similarity module, an interaction analysis performed at the monitoring device, and a configured aggregation level of sensor data. The sensor data indicates a brain activity of one or more regions in a brain of the user. The network node initiates an action related to an experience of the user based on the received aggregated activity graph.

It is furthermore provided herein a computer program product comprising instructions, which, when executed on at least one processor, cause the at least one processor to carry out any of the methods herein, as performed by the monitoring device and the network node, respectively. It is additionally provided herein a computer-readable storage medium, having stored thereon a computer program product comprising instructions which, when executed on at least one processor, cause the at least one processor to carry out any of the methods herein, as performed by the monitoring device and the network node, respectively.

According to yet another aspect the object may be achieved by providing a monitoring device for handling brain activity data of a user. The monitoring device is configured to obtain sensor data from a plurality of sensors, wherein the sensor data indicates a brain activity of one or more regions in a brain of the user. The monitoring device is further configured to construct a similarity module of brain activities based on an activity configuration defining one or more sensors out of the plurality of sensors to gather sensor data from, and obtained sensor data from the one or more sensors, wherein the similarity module comprises one or more groups of sensor data related to one another. The monitoring device is configured to perform an interaction analysis within and/or between the one or more groups in the similarity module by creating a network of interactions within and/or between the one or more groups. The monitoring device is further configured to create an aggregated activity graph based on the constructed similarity module, the performed interaction analysis, and a configured aggregation level of sensor data; and to provide the aggregated activity graph to a network node.

According to still another aspect the object may be achieved by providing a network node for handling brain activity data of a user. The network node is configured to receive from a monitoring device related to the user, an aggregated activity graph, wherein the aggregated activity graph is based on a similarity module, an interaction analysis performed at the monitoring device, and a configured aggregation level of sensor data. The sensor data indicates a brain activity of one or more regions in a brain of the user. The network node is further configured to initiate an action related to an experience of the user based on the received aggregated activity graph.

The aggregated activity graph is a compact representation of brain activity providing a privacy-preserving solution and/or a resource efficient way of communicating the brain activity.

### BRIEF DESCRIPTION OF THE DRAWINGS

Embodiments will now be described in more detail in relation to the enclosed drawings, in which:

FIG. 1 shows an immersive session that may involve a number of senses according to prior art;

FIG. 2 shows a communication network according to embodiments herein;

FIG. 3 shows a schematic combined flowchart and signalling scheme according to embodiments herein;

FIG. 4 shows a flowchart depicting a method performed by a monitoring device according to embodiments herein;

FIG. 5 shows a flowchart depicting a method performed by a network node according to embodiments herein;

FIG. 6 shows a flowchart depicting a method performed by a monitoring device according to embodiments herein;

FIG. 7 shows a flow diagram of modules performing actions according to embodiments herein;

FIG. 8 shows modules performing actions according to embodiments herein;

FIG. 9 shows a schematic overview for obtaining sensor data according to embodiments herein;

FIG. 10 shows a schematic overview for constructing a similarity matrix according to embodiments herein;

FIG. 11a shows a schematic overview for performing interaction analysis according to embodiments herein;

FIG. 11b shows a schematic overview for creating an aggregated activity graph according to embodiments herein;

FIG. 12 shows a schematic overview depicting a way of creating a reference interaction network according to embodiments herein;

FIGS. 13a-b show schematic overviews depicting a monitoring device according to embodiments herein;

FIGS. 14a-b show schematic overviews depicting a network node according to embodiments herein;

FIG. 15 schematically illustrates a telecommunication network connected via an intermediate network to a host computer;

FIG. 16 is a generalized block diagram of a host computer communicating via a base station with a user equipment over a partially wireless connection; and

FIGS. 17-20 are flowcharts illustrating methods implemented in a communication system including a host computer, a base station and a user equipment.

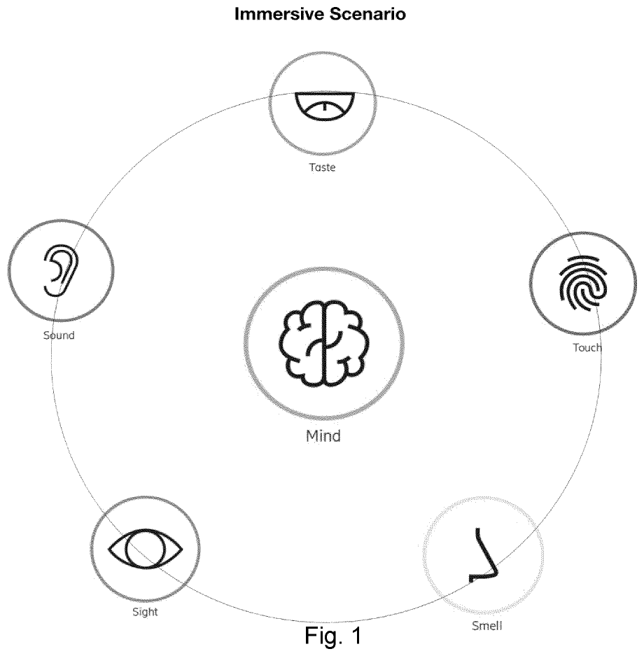

### DETAILED DESCRIPTION

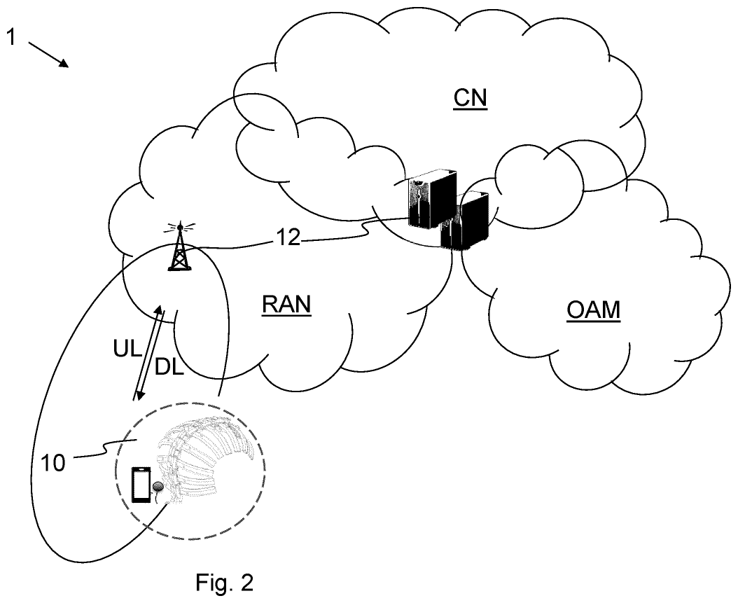

Embodiments herein relate to communication networks in general. FIG. 2 is a schematic overview depicting a communication network 1 handling packet communication, for example, a network associated with a cloud infrastructure. The communication network 1 may comprise one or more access networks, such as radio access networks (RAN) connected to one or more core networks (CN). The communication network 1 may further comprise an operation, administration, and maintenance (OAM) network. The communication network 1 may use a number of different technologies, such as an optical network, a wired network, an IP network, a wireless network such as Wi-Fi, Long Term Evolution (LTE), LTE-Advanced, New Radio (NR), Wideband Code Division Multiple Access (WCDMA), Global System for Mobile communications/Enhanced Data rate for GSM Evolution (GSM/EDGE), Worldwide Interoperability for Microwave Access (WiMax), or Ultra Mobile Broadband (UMB), as well as future mobile network access technologies just to mention a few possible implementations.

In the communication network 1, a monitoring device 10 is comprised for collecting sensor data indicating brain activity of one or more regions in a brain of the user brain activity. The sensor data may be collected from a plurality of sensors. The monitoring device 10 may comprise one or more sensors or be a separate unit separated from the plurality of sensors. The monitoring device 10 may be a device capable of electroencephalography, i.e., a device recording electrical activities of the brain.

The communication network may further comprise a network node 12.

The network node 12 may be a network element that provides network connectivity to, or is connected to, a terminal device including user equipment (UE), internet of things (IoT) sensor or actuator device. The network node 12 may be a server, a wireless or wired node, a radio base station, WiFi access router, an OAM node, or similar.

According to embodiments herein the monitoring device 10 constructs a similarity module of brain activities based on an activity configuration defining one or more sensors out of the plurality of sensors to gather sensor data from, also referred to as an activity map, and obtained sensor data from the one or more sensors. The similarity module such as a similarity matrix, comprises one or more groups of sensor data related to one another. For example, a block-matrix where the diagonal elements are square matrices containing regional similarities within senses and off-diagonal elements contain regional similarities in-between senses. The monitoring device 10 performs an interaction analysis within and/or between the one or more groups in the similarity module by creating a network of interactions within and/or between the one or more groups.

The monitoring device 10 then creates an aggregated activity graph based on the constructed similarity module, the performed interaction analysis, and a configured aggregation level of sensor data. This aggregated activity graph is then provided to the network node 12. The network node 12 may then perform an action based on the aggregated activity graph such as order transmission of, or transmit, content based on the aggregated activity graph.

It is thus herein provided a process for an extraction of privacy-preserving brain-activity-related features for targeted content recommendation and generation systems. The method creates the aggregated activity graph, also referred to as an aggregated activity map or a privacy-preserving brain sensory activity map that carries aggregated information about senses and interaction within and in-between the senses. Thus, the aggregated activity graph may include both nodes and connections between brain regions. The aggregated activity graph may be available in at least two aggregation levels: At a first level, the aggregated activity graph carries high resolution information about the user's brain sensory interactions. At this level the map may be a square matrix of a size equal to a number of brain regions monitored at the immersive session. If desired, it may be secured in a cost-effective way due to fairly compact structure of the map. At the second level, the aggregated activity graph may take on a highly aggregated form which is fully privacy-preserving with a compact size presented by a square matrix of a size equal to the number of senses, such as a 5×5-dimensional matrix corresponding to five senses. The aggregated activity graph is thus a compact representation providing a privacy-preserving solution and/or a resource efficient way of communicating the brain activity. Thus, it is herein provided one or more methods for extraction of privacy-preserving brain-activity-related features in an immersive scenario involving multiple senses, named aggregated activity graph or aggregated brain activity map, which carries highly aggregated information about, for example, senses and interaction within and in-between the senses.

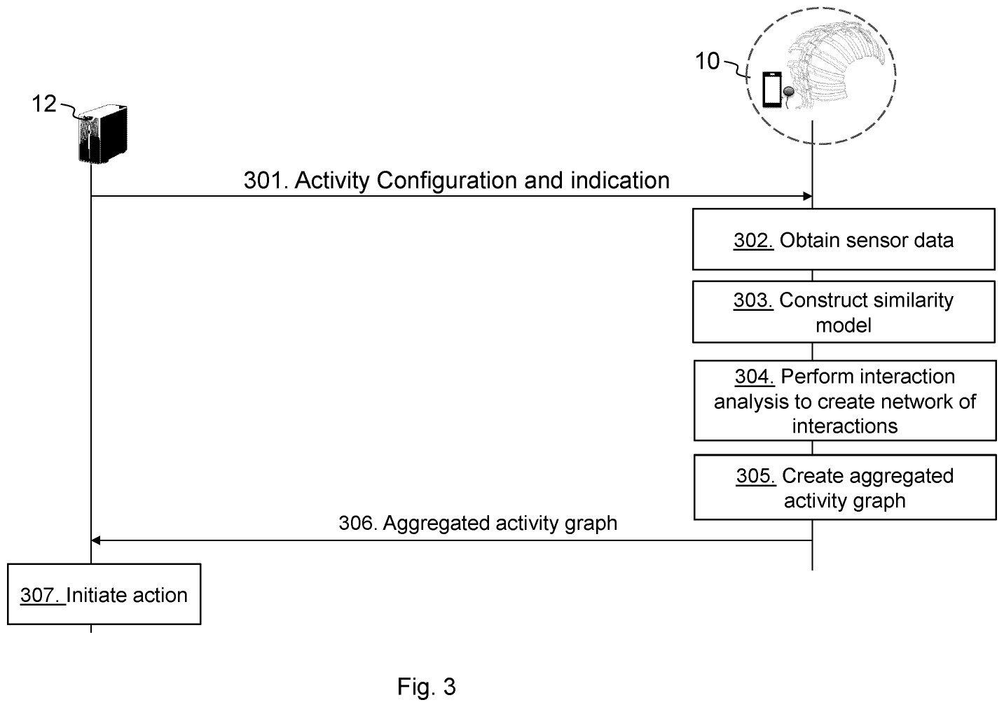

FIG. 3 is a combined flowchart and signalling scheme depicting embodiments herein.

Action 301. The network node 12 may transmit an activity configuration defining one or more sensors to gather sensor data from and an indication of an aggregation level (of the aggregated activity graph).

Action 302. The monitoring device 10 may obtain sensor data from sensors, for example, as indicated by the activity configuration or from all sensors.

Action 303. The monitoring device 10 constructs the similarity module of brain activities based on the activity configuration, and the obtained sensor data from the one or more sensors. The similarity module comprises one or more groups of sensor data related to one another.

Action 304. The monitoring device 10 performs the interaction analysis within and/or between the one or more groups in the similarity module by creating the network of interactions within and/or between the one or more groups.

Action 305. The monitoring device 10 further creates the aggregated activity graph based on the constructed similarity module, the performed interaction analysis and the aggregation level, for example, as indicated by the indication in action 301.

Action 306. The monitoring device 10 transmits the aggregated activity graph. The aggregated activity graph may be referred to as aggregated activity map and provides privacy-preserving features from sensor data of brain activity, which are by construction privacy preserving. The compact size of the aggregated activity graph facilitates an efficient usage of communication resources when communicating this information. Communication resources herein meaning bandwidth, frequency and/or time slots. Embodiments herein allow for at least a two level of aggregation. At a first level, the aggregated activity graph is larger in size but carries a high resolution description of the brain activities. At this level the aggregated activity graph is less privacy preserving. At a second level, a highly aggregated activity graph is extracted which is compact in size and more privacy-preserving by nature.

Action 307. The network node 12, receiving the aggregated activity graph, initiates an action based on the aggregated activity graph. For example, the network node 12 may order transmission of, or transmit, content based on the aggregated activity graph.

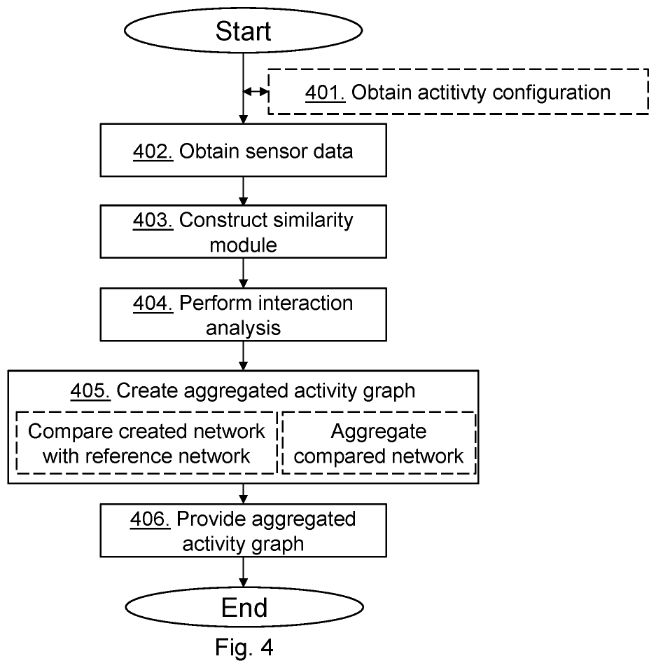

The method actions performed by the monitoring device 10 for handling, for example, processing, brain activity data of a user according to embodiments herein will now be described with reference to a flowchart depicted in FIG. 4. The actions do not have to be taken in the order stated below, but may be taken in any suitable order. Actions performed in some embodiments are marked with dashed boxes. The monitoring device 10 may be related to the user, for example, attached or connected to a sensor arrangement or comprising one or more sensors.

Action 401. The monitoring device 10 may obtain the activity configuration and an indication of the configured aggregation level of sensor data from within the monitoring device 10, or from the network node 12, for example, a server entity, such as content provider or a recommender system. Thus, the activity configuration may be retrieved from a memory or be received from the network node 12. The activity configuration may comprise an activity map or activity list and the aggregation level. The monitoring device 10 may receive from the network node 12 (i) a reference brain sensory activity map, (ii) a list of the brain regions to be monitored (the regions are selected to be reflective of the immersive scenario presented to the user), (iii) a required aggregation level. The monitoring device 10 may receive a reference network of interactions from the network node 12. It should be noted that the reference network of interactions or an updated reference network of interactions may be received after action 406.

Action 402. The monitoring device 10 obtains sensor data from a plurality of sensors, wherein the sensor data indicates a brain activity of one or more regions in a brain of the user, also referred to as brain sensory regions. The sensor data may be pushed from the sensors to the monitoring device 10 or retrieved from within. Sensor data may comprise one or more values of readings indicating electrical activities, energy readings, or similar.

Action 403. The monitoring device 10 constructs the similarity module of brain activities based on the activity configuration defining the one or more sensors out of the plurality of sensors to gather sensor data from, and the obtained sensor data from the one or more sensors. The similarity module comprises one or more groups of sensor data related to one another. For example, the monitoring device 10 may compute a similarity matrix from brain regional activities, also referred to as a brain similarity matrix. Given measured brain activities from selected brain sensory regions, the monitoring device 10 may construct a similarity matrix, which similarity matrix quantifies the similarity of the regional brain activities. Elements of the similarity matrix describes the degree of similarity between two regions.

Action 404. The monitoring device 10 performs the interaction analysis within and/or between the one or more groups in the similarity module by creating the network of interactions within and/or between the one or more groups. The one or more sensors may be related to one or more regions of the brain to monitor, and the similarity module may comprise the similarity matrix constructed from sensor data of the one or more regions. The network of interactions may then be computed by performing community analysis on the similarity matrix. The community analysis discovers the community structure within the network of brain regions/senses. For example, the monitoring device 10 may extract a brain sensory interaction network by, from the similarity matrix, extract the network of interactions between brain sensory regions containing community structures of statistically most significant interactions in-between and within brain sensory regions.

Action 405. The monitoring device 10 creates the aggregated activity graph based on the constructed similarity module, the performed interaction analysis, and the configured aggregation level of sensor data. The monitoring device 10 may create the aggregated activity graph by comparing the created network of interactions with a reference network of interactions and by forming the aggregated activity graph based on said comparison. Thus, the aggregated activity graph may be a representation based on the comparison. Furthermore, a compared network of interactions may be obtained from the comparison and the monitoring device 10 may create the aggregated activity graph by further constructing the aggregated activity graph through aggregating the compared network of interactions into the aggregated activity graph.

The monitoring device 10 may, for example, construct a user's personalized aggregated activity graph from the user brain activities at an immersive session based on the requirements (i) to (iii) in action 401. As an example, the monitoring device 10 may extract the aggregated activity graph, also referred to as aggregated activity map, from the sensor data. Based on the network of interaction, the monitoring device 10 may construct an aggregated matrix of brain sensory interactions which carry high level information about the interactions in-between and within the senses.

Action 406. The monitoring device 10 further provides the aggregated activity graph to the network node 12. The monitoring device 10 may transmit the aggregated activity graph to the network node 12 or the aggregated activity graph may be pulled from the network node 12.

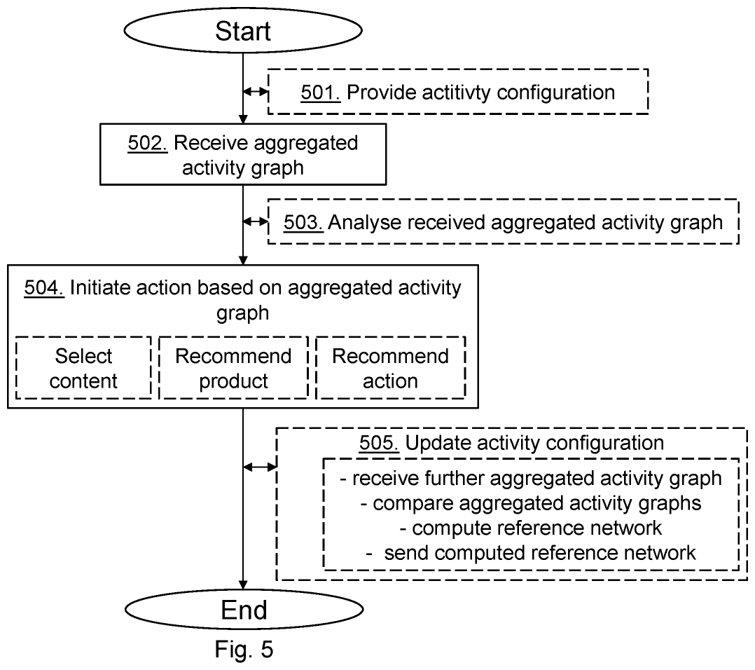

The method actions performed by the network node 12 for handling, for example, processing, brain activity data of the user according to embodiments herein will now be described with reference to a flowchart depicted in FIG. 5. The actions do not have to be taken in the order stated below, but may be taken in any suitable order. Actions performed in some embodiments are marked with dashed boxes.

Action 501. The network node 12 may provide to the monitoring device 10, the activity configuration defining the one or more sensors out of the plurality of sensors to gather sensor data from, and the indication of the configured aggregation level of sensor data. The indication may be a value or an index.

Action 502. The network node 12 receives from the monitoring device 10 related to the user, the aggregated activity graph. The aggregated activity graph being based on the similarity module, the interaction analysis performed at the monitoring device 10, and a configured aggregation level of sensor data. The sensor data indicates the brain activity of one or more regions in the brain of the user.

Action 503. The network node 12 may analyse the received aggregated activity graph. For example, the network node 12 may determine to perform, or initiate, an action based on the received aggregated activity graph. The network node 12 may, for example, compare the received aggregated activity graph with a threshold or similar.

Action 504. The network node 12 initiates the action related to an experience of the user based on the received aggregated activity graph. Thus, the network node 12 may associate the user's current brain activity to the aggregated activity graph and based on that perform an action. The action initiated may comprise one or more of the following:
selecting content to provide to the user based on the received aggregated activity graph. For example, during a generative gaming experience, upcoming scenes may be generated and selected based on the aggregated activity graph. If the aggregated activity graph indicates that the user is bored, i.e., electrical readings below a threshold in certain areas, more exciting content may be generated. In another example, content may be recommended to balance out any unwanted cases such as fear by recommending less scary content or sadness by recommending something happier. This may also depend on a user's context and not just their brain activity;
recommending a product or a service to the user based on the received aggregated activity graph;
recommending an action for a third party to perform based on the received aggregated activity graph.

Action 505. The network node 12 may update the activity configuration based on one or more received aggregated activity graphs. For example, the network node 12 may receive a further aggregated activity graph from another monitoring device. The network node 12 may compare the received aggregated activity graph and the further aggregated activity graph to group aggregated activity graphs of users. The network node 12 may further compute a reference network of interactions for the users of the grouped aggregated activity graphs; and send the computed reference network of interactions to monitoring devices of the users.

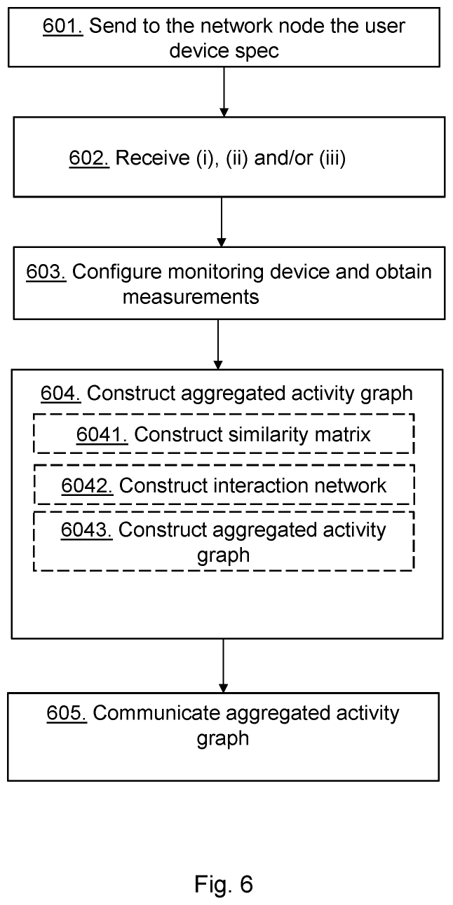

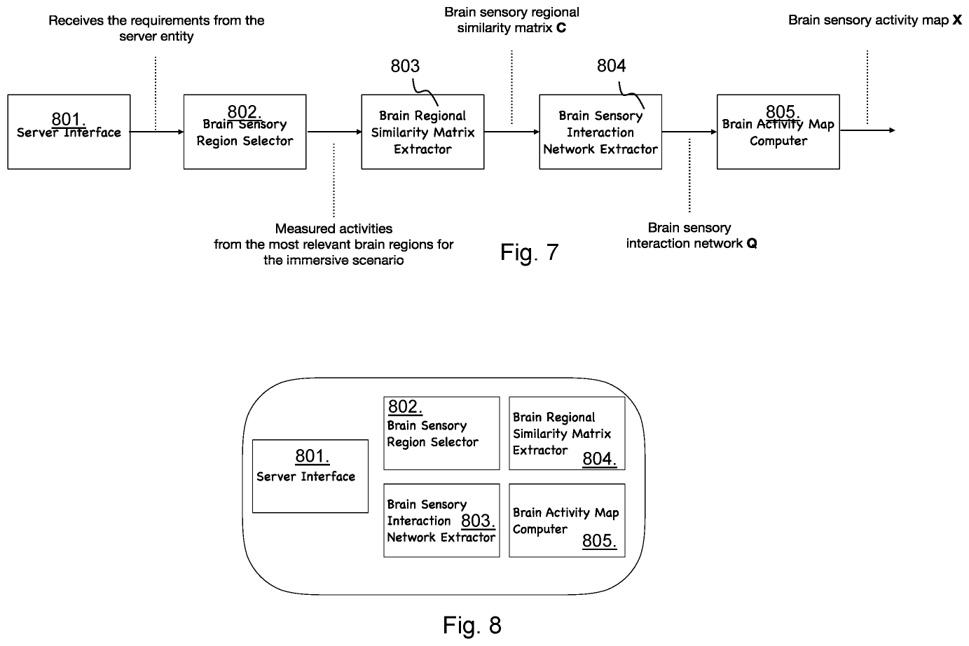

According to an exemplary method of an apparatus it is herein described the following steps and presented with reference to FIG. 6 and FIG. 7.

At the monitoring device 10:

Action 601. Communication with the network node 12 about the user device specifications via the Server Interface module.
Information about user device specifications can be used to form distinct clusters where each cluster contains users who share the same infrastructure. This would allow the network node 12 to aggregate all users whose data are collected with one type of a device to one cluster and another set of users with another one.

Action 602. Receive the following requirements from the network node 12 via Server Interface module:
(i) a reference brain sensory activity map,
(ii) a list of the brain regions to be monitored (the regions are for example selected to be reflective of the immersive scenario presented to the user),
(iii) the required aggregation level.

Action 603. Configuring the monitoring device 10 according to the list of selected brain regions from the previous step via Brain Sensory Region Selector 802. Obtaining measurements from the identified brain regions.

Action 604. Construction of the aggregated activity graph or map by applying one or more of the following steps:

Action 6041. Construction of the similarity matrix by application of a first module denoted Brain Regional Similarity Matrix Extractor 803.

Action 6042. Construction of the network of interaction by application of a second module denoted Brain Sensory Interaction Network Extractor 804.

Action 6043. Construction of the aggregated activity graph by application of a third module denoted Brain Sensory Activity Map Computer 805.

Action 605. Communication of the aggregated activity graph with the network node 12 via Server Interface 801. If the aggregation level from action 602 is level 1, the network of interaction from action 6042 is communicated and if the aggregation level is 2, the aggregated activity graph from step 6043 is communicated.

### On the Network Node 12

The network node 12 such as a content provider or a server entity, may construct communities from users aggregated activity graphs. Users with similar aggregated activity graphs are grouped together and receive similar recommendations, or contents are generated such that suit them best. The personalized contents are sent to the users.

### Components of the Monitoring Device 10

Examples of components of the apparatus are shown in FIGS. 7 and 8 and described in this section in details.

### Server Interface 801

Via the server interface 801 the monitoring device 10 communicates with the network node 12. The network node 12 can for example be a content provider or a recommendation system.

### Brain Sensory Region Selector 802

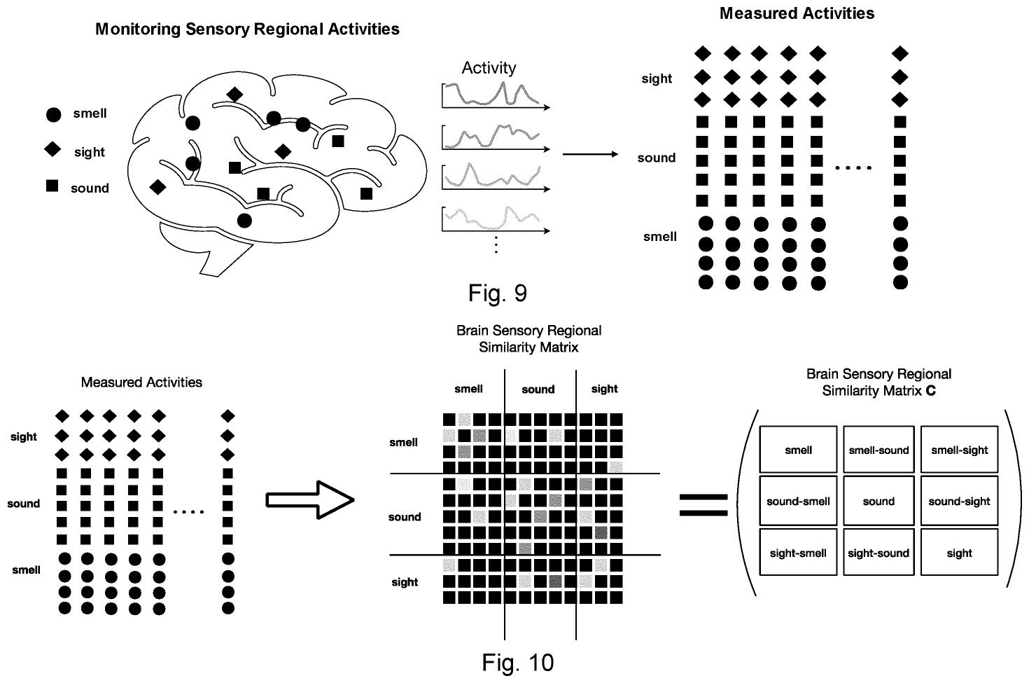

The Brain Sensory Region Selector 802 selects the brain regions corresponding to the immersive scenario presented to the user. For example, in an immersive scenario involving three senses of smell, sight (vision), and sound (hearing), only those regions are selected that are specialized for these senses. Refer to FIG. 9 for the visualization. This is an example of action 402.

FIG. 9 shows that the monitoring device 10 monitors brain activities corresponding to the regions identified by the Brain Sensory Region Selector. These regions may be selected to be of outmost relevance to the immersive scenario presented to the users. In the example shown here, these are the three senses of smell, sight, sound.

### Brain Regional Similarity Matrix Extractor 803

Given the measured brain activities from selected brain regions, a similarity matrix is constructed. The similarity matrix quantifies the similarity of the regional brain activities. For R regions, the resulting matrix, named C, is an (R×R)-dimensional matrix where each element of the matrix, Cᵢⱼ The total number of regions is given by R=ΣₖRₖ where Rₖ is the number of regions corresponding to the sense k. FIG. 10 shows an example of a similarity matrix for an immersive scenario involving three senses of sight, smell, sound. shows an example of a similarity matrix for an immersive scenario involving three senses of sight, smell, sound. FIG. 10 shows that the similarity matrix is constructed from measured brain activities. The resulting matrix C can be seen as a block-matrix where the diagonal elements are square matrices containing regional similarities within senses and off-diagonal elements contain regional similarities in-between senses. This is an example of action 403.

Implementation: An example implementation of this module is via constructing the partial correlation matrix computed from the covariance matrix of the brain activities. Another possible implementation is using cosine similarity as the choice of distance metric for computing the similarities.

### Brain Sensory Interaction Network Extractor 804

Given the brain regional similarity matrix C, the Brain Sensory Interaction Network Extractor 804 extracts a network of interactions between brain regions, named brain sensory interaction network. The network of interaction contains community structures of statistically most significant interactions. The network of interaction contains K communities where K is equal to the number of senses presented in the immersive scenario. The network of interaction summarizes two types of interactions, namely intra-community interactions and inter-community interactions. The intra-community interactions are the interactions within the communities, and inter-interactions are the interactions in-between the communities. The network of interaction may be expressed by a (K×K)-dimensional block-matrix Q where each element on the diagonal of Q contains the matrix of intra-community interactions. The off-diagonal elements contain the inter-community interactions between the senses. This is an example of action 404.

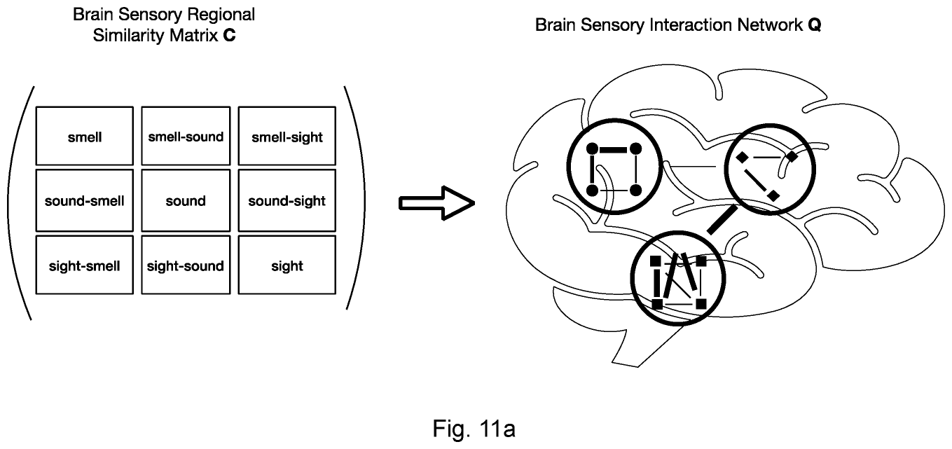

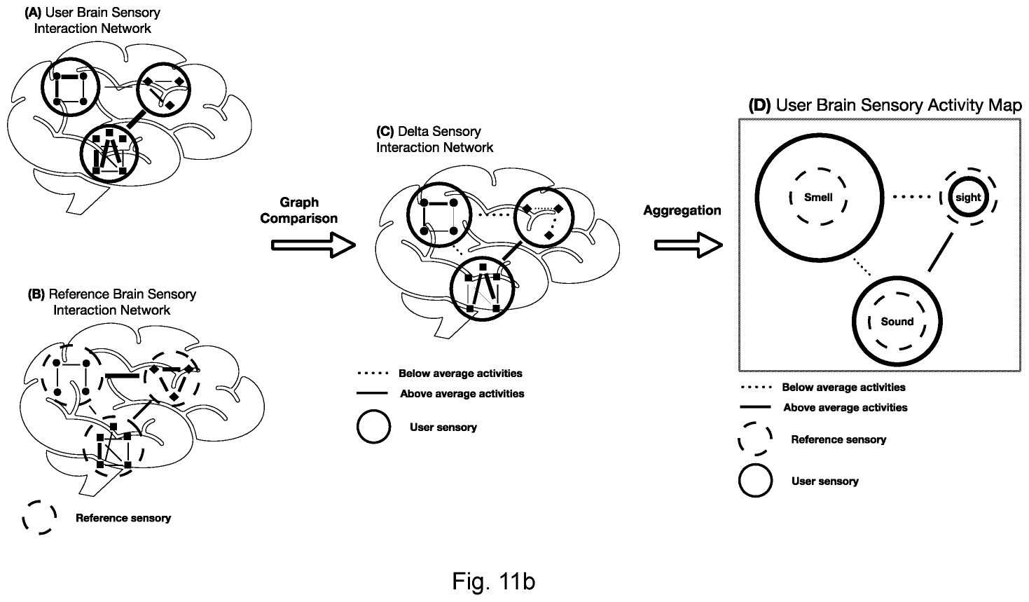

The intra-community interactions represent the intra-sensory regional interactions and can be effectively expressed by graphs with weighted edges. The inter-community interactions represent the inter-sensory interactions. Both inter and intra sensory interaction graphs are sparse graphs containing interactions with statistical significance. FIG. 11a shows a network of interaction for an immersive scenario involving three senses of sight, smell, sound. FIG. 11a shows that the network of interaction is computed by performing community analysis on the similarity matrix. The network of interaction, also referred to as brain sensory interaction network, is represented by a number of communities K equal to the number of senses presented in the immersive scenario. Each community represents a sense. The intra-community interactions represent the intra-sensory interactions and are expressed by graphs with weighted edges. The inter-community interactions represent the inter-sensory interactions. Both inter and intra sensory graphs are sparse graphs where the sparsity may be enforced by only keeping interactions with statistical significance.

An example implementation of this module is via Louvain community detection method applied on the brain regional similarity matrix. The community detection is performed on the condition that the number of communities K is known.

### Brain Sensory Activity Map Computer 805

Given the matrix of sensory interaction network Q, the module constructs an aggregated matrix of brain sensory interactions, expressed by a (K×K)-dimensional matrix X. This is an example of action 405.

The diagonal elements of the matrix, Xᵢᵢ it contains aggregated information about the intra-sensory interactions, and it is computed as:

$${X}_{ii} = average ( {Δ}_{ii} ) , {Δ}_{ii} = d ( {Q}_{ii} , {\overline{Q}}_{ii} ) ) . \qquad ( 1 )$$

In this equation, Q̅ᵢᵢ is the reference sensory interaction network, provided by the network node 12 to all users, and d (·,·) measures the deviation of the user sensory interactions matrix Qᵢᵢ from the reference Q̅ᵢᵢ. The resulting matrix Δᵢᵢ indicates how different is the user sensory interactions from expected quantity. The operator average (·) takes the average of Δᵢᵢ and outputs a scalar value.

The off-diagonal elements Xᵢⱼ contains aggregated information about the inter-sensory interactions, and it is computed as:

$${X}_{ij} = average ( {Δ}_{ij} ) , {Δ}_{ij} = d ( {Q}_{ij} , {\overline{Q}}_{ij} ) ) . \qquad ( 2 )$$

Implementation: The distance d(Qᵢᵢ, Q̅ᵢᵢ) can simply be the element-wise difference between the two corresponding elements from matrices Q and Q̅. It can alternatively be computed by representing Q and Q̅ by graphs and comparing the two graphs. The latter would consider the dependencies between the regions while the former would discard such dependencies. The network of interaction may, as a first level of aggregation, be the aggregated activity graph.

FIG. 11b shows that the aggregated activity graph of the user may be computed from the reference interaction network Q̅ and the interaction network Q. (A) The network of interaction or the user sensory interaction network Q. (B) The reference network of interaction or reference brain sensory interaction network Q̅. (C) The difference (delta) network Δ which quantifies how much user network Q differs from the expected average Q̅, computed as Δ=d(Q̅, Q). The lines color-coded by dotted lines indicates that the interactions are lower than the average while the black color-coded lines indicate that the interactions are larger than average. The thickness of the line indicates the strength. (D) The aggregated activity graph or user brain sensory activity map may be represented by a graph. In FIG. 11b, the average level of activities is shown by dotted circle while the ones for the user are shown with solid-line circles. The size of the user circle would indicate how much lower or higher the aggregated level of activity is from the average quantity (See equations (1) and (2)). The dotted lines indicates that the intra-sensory interactions are lower than the average while the black color-coded lines indicate that the interactions are larger than average. The thickness of the line indicates the strength. The user brain activity map can be formally represented by a graph where nodes indicate the aggregated inter-sensory activities in comparison to the reference and edges indicate the intra-sensory activities in comparison to the reference.

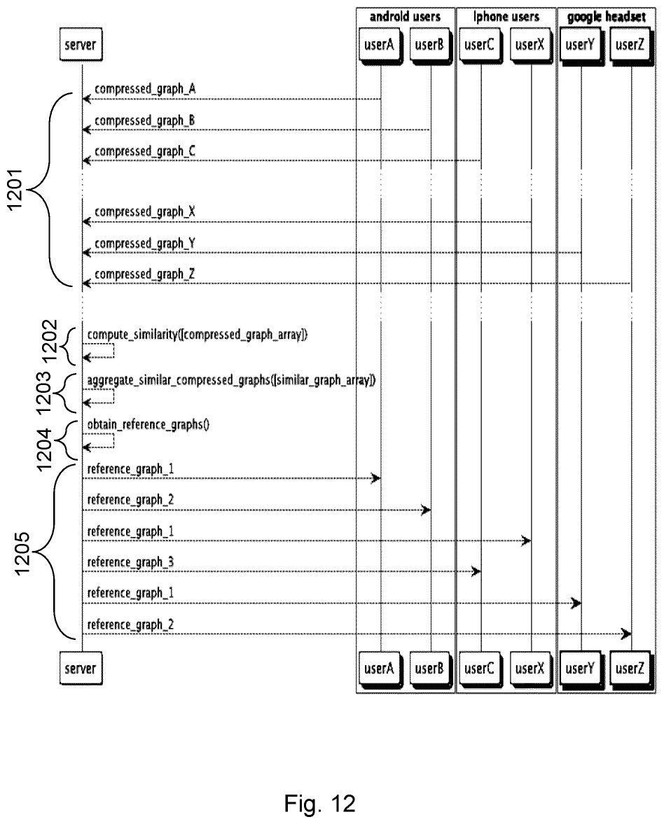

Below an implementation is described for construction of the reference activity graph on the network node 12. FIG. 12 shows an implementation to obtain the reference network of interaction at the network node 12.

The network node 12 receives, see action 1201, aggregated activity graphs from users, also referred to as compressed graphs, and may perform, see action 1202, first similarity to group the similar ones. Once the grouping is performed, the aggregation of the graphs is performed, see action 1203, to compute the expected reference graph for every user group, see action 1204. The reference graphs are then sent to each user, see action 1205. Multiple users might have the same expected reference graph. This is sketched via a signal flow diagram in FIG. 12.

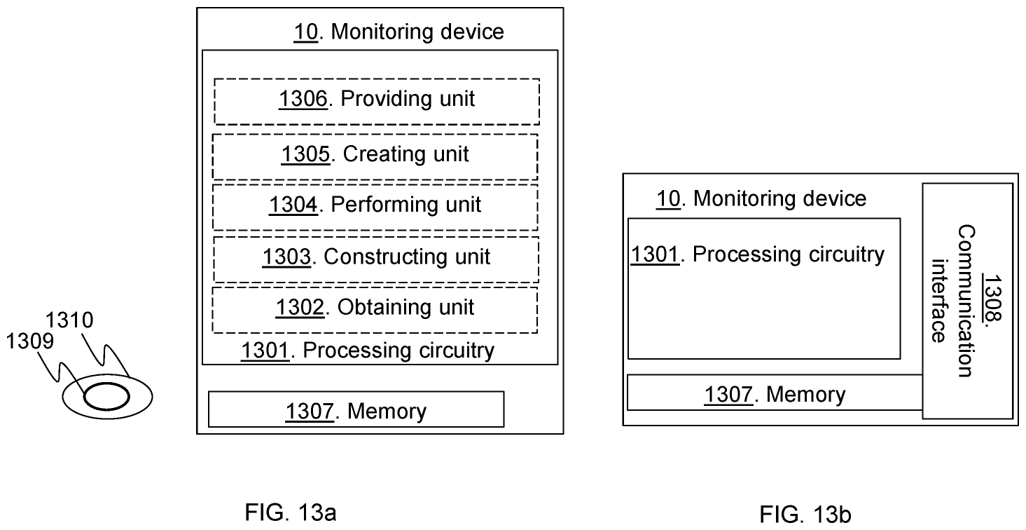

FIGS. 13a-13b are schematic overview depicting the monitoring device 10 for handling brain activity data of a user according to embodiments herein.

The monitoring device 10 may comprise processing circuitry 1301, such as one or more processors, configured to perform methods herein. The processing circuitry may be arranged in one stand-alone unit or be distributed among a number of servers or units.

The monitoring device 10 may comprise an obtaining unit 1302 such as a receiver, a transceiver, a reader or similar. The monitoring device 10, the processing circuitry 1301, and/or the obtaining unit 1302 is configured to obtain the sensor data from the plurality of sensors, wherein the sensor data indicates the brain activity of the one or more regions in the brain of the user. The monitoring device 10, the processing circuitry 1301, and/or the obtaining unit 1302 may be configured to obtain the activity configuration and the indication of the configured aggregation level of sensor data from within the monitoring device 10 or from the network node 12.

The monitoring device 10 may comprise a constructing unit 1303 such as a computing unit or similar. The monitoring device 10, the processing circuitry 1301, and/or the constructing unit 1303 is configured to construct the similarity module of brain activities based on the activity configuration defining the one or more sensors out of the plurality of sensors to gather sensor data from, and the obtained sensor data from the one or more sensors. The similarity module comprises the one or more groups of sensor data related to one another.

The monitoring device 10 may comprise a performing unit 1304 such as a computing unit or similar. The monitoring device 10, the processing circuitry 1301, and/or the performing unit 1304 is configured to perform the interaction analysis within and/or between the one or more groups in the similarity module by creating the network of interactions within and/or between the one or more groups. The one or more sensors are related to one or more regions of the brain to monitor, and the similarity module comprises a similarity matrix constructed from sensor data of the one or more regions, and the monitoring device 10, the processing circuitry 1301, and/or the performing unit 1304 may be configured to compute the network of interactions by performing community analysis on the similarity matrix.

The monitoring device 10 may comprise a creating unit 1305 such as a computing unit or similar. The monitoring device 10, the processing circuitry 1301, and/or the creating unit 1305 is configured to create the aggregated activity graph based on the constructed similarity module, the performed interaction analysis, and the configured aggregation level of sensor data. The monitoring device 10, the processing circuitry 1301, and/or the creating unit 1305 may be configured to create the aggregated activity graph by comparing the created network of interactions with the reference network of interactions and forming the aggregated activity graph based on said comparison. The compared network of interactions may be obtained from the comparison and the monitoring device 10, the processing circuitry 1301, and/or the creating unit 1305 may be configured to create the aggregated activity graph by aggregating the compared network of interactions into the aggregated activity graph.

The monitoring device 10 may comprise a providing unit 1306 such as a transmitter, a transceiver, a writer or similar. The monitoring device 10, the processing circuitry 1301, and/or the providing unit 1306 is configured to provide the aggregated activity graph to the network node 12.

The monitoring device 10, the processing circuitry 1301, and/or the obtaining unit 1302 may be configured to receive the reference network of interactions from the network node 12.

The monitoring device 10 comprises a memory 1307. The memory 1307 comprises one or more units to be used to store data on, such as indications, similarity module, network of interaction, aggregated activity graph, activity configuration, action, resource information, data related to sensors, and applications to perform the methods disclosed herein when being executed, and similar. Thus, embodiments herein may disclose monitoring device 10 for handling brain activity data of the user, wherein the monitoring device 10 comprises processing circuitry and a memory, said memory comprising instructions executable by said processing circuitry whereby said monitoring device 10 is operative to perform any of the methods herein. Furthermore, the arrangement 13 may comprise a communication interface 1308 comprising, e.g., a transmitter, a receiver, a transceiver and/or one or more antennas.

The methods according to the embodiments described herein for the monitoring device 10 are respectively implemented by means of, e.g., a computer program product 1309 or a computer program, comprising instructions, i.e., software code portions, which, when executed on at least one processor, cause the at least one processor to carry out the actions described herein, as performed by the monitoring device 10. The computer program product 1309 may be stored on a computer-readable storage medium 1310, e.g., a disc, a universal serial bus (USB) stick or similar. The computer-readable storage medium 1310, having stored thereon the computer program product, may comprise the instructions which, when executed on at least one processor, cause the at least one processor to carry out the actions described herein, as performed by the monitoring device 10. In some embodiments, the computer-readable storage medium may be a transitory or a non-transitory computer-readable storage medium.

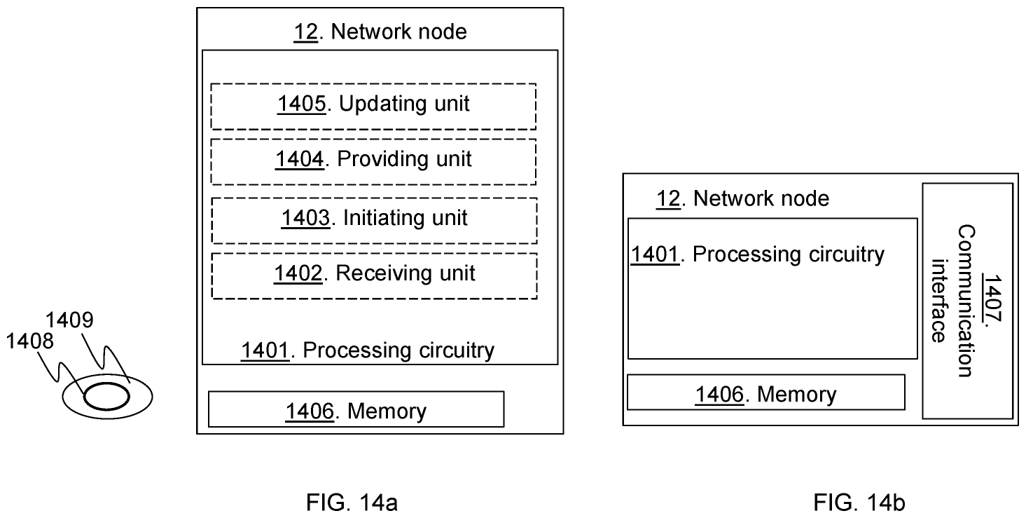

FIGS. 14a-b are schematic overviews are schematic overview depicting the network node 12 for handling brain activity data of a user according to embodiments herein.

The network node 12 may comprise processing circuitry 1401, such as one or more processors, configured to perform methods herein. The processing circuitry 1401 may be arranged in one stand-alone unit or be distributed among a number of servers or units.

The network node 12 may comprise a receiving unit 1402 such as a receiver, a transceiver, a reader or similar. The network node 12, the processing circuitry 1401, and/or the receiving unit 1402 is configured to receive from the monitoring device 10 related to the user, the aggregated activity graph, wherein the aggregated activity graph is based on the similarity module, the interaction analysis performed at the monitoring device, and the configured aggregation level of sensor data. The sensor data indicates the brain activity of the one or more regions in the brain of the user.

The network node 12 may comprise an initiating unit 1403 such as a trigger unit, selector, a reader or similar. The network node 12, the processing circuitry 1401, and/or the initiating unit 1403 is configured to initiate the action related to the experience of the user based on the received aggregated activity graph. The action initiated may comprise one or more of the following:
selecting content to provide to the user based on the received aggregated activity graph;
recommending the product or the service to the user based on the received aggregated activity graph;
recommending the action for a third party to perform based on the received aggregated activity graph.

The network node 12 may comprise a providing unit 1404 such as a transmitter, a transceiver, a writer or similar. The network node 12, the processing circuitry 1401, and/or the providing unit 1404 may be configured to provide to the monitoring device, the activity configuration defining the one or more sensors out of the plurality of sensors to gather sensor data from, and the indication of the configured aggregation level of sensor data.

The network node 12 may comprise an updating unit 1405 such as a computing unit, a comparator, or similar. The network node 12, the processing circuitry 1401, and/or the updating unit 1405 may be configured to update the activity configuration based on one or more received aggregated activity graphs. The network node 12, the processing circuitry 1401, and/or the updating unit 1405 may be configured to receive a further aggregated activity graph from another monitoring device; compare the received aggregated activity graph and the further aggregated activity graph to group aggregated activity graphs of users; compute a reference network of interactions for the users of the grouped aggregated activity graphs; and send the computed reference network of interactions to monitoring devices of the users.

The network node 12 comprises a memory 1406. The memory 1406 comprises one or more units to be used to store data on, such as indications, similarity module, network of interaction, aggregated activity graph, activity configuration, action, resource information, data related to sensors, and applications to perform the methods disclosed herein when being executed, and similar. Thus, embodiments herein may disclose a network node 12 for handling brain activity data of the user, wherein the network node 12 comprises processing circuitry and a memory, said memory comprising instructions executable by said processing circuitry whereby said network node 12 is operative to perform any of the methods herein. Furthermore, the network node 12 may comprise a communication interface 1407 comprising, e.g., a transmitter, a receiver, a transceiver and/or one or more antennas.

The methods according to the embodiments described herein for the network node 12 are respectively implemented by means of e.g. a computer program product 1408 or a computer program, comprising instructions, i.e., software code portions, which, when executed on at least one processor, cause the at least one processor to carry out the actions described herein, as performed by the network node 12. The computer program product 1408 may be stored on a computer-readable storage medium 1409, e.g., a disc, a universal serial bus (USB) stick or similar. The computer-readable storage medium 1409, having stored thereon the computer program product, may comprise the instructions which, when executed on at least one processor, cause the at least one processor to carry out the actions described herein, as performed by the network node 12. In some embodiments, the computer-readable storage medium may be a transitory or a non-transitory computer-readable storage medium.

In some embodiments a more general term “network node” is used and it can correspond to any type of radio-network node or any network node, which communicates with a wireless device and/or with another network node. Examples of network nodes are NodeB, MeNB, SeNB, a network node belonging to Master cell group (MCG) or Secondary cell group (SCG), base station (BS), multi-standard radio (MSR) radio node such as MSR BS, eNodeB, network controller, radio-network controller (RNC), base station controller (BSC), relay, donor node controlling relay, base transceiver station (BTS), access point (AP), transmission points, transmission nodes, Remote radio Unit (RRU), Remote Radio Head (RRH), nodes in distributed antenna system (DAS), etc.

In some embodiments the non-limiting term wireless device or user equipment (UE) is used and it refers to any type of wireless device communicating with a network node and/or with another wireless device in a cellular or mobile communication system. Examples of UE are target device, device to device (D2D) UE, proximity capable UE (aka ProSe UE), machine type UE or UE capable of machine to machine (M2M) communication, Tablet, mobile terminals, smart phone, laptop embedded equipped (LEE), laptop mounted equipment (LME), USB dongles etc.

Embodiments are applicable to communication technology such as any RAT or multi-RAT systems, where the UE receives and/or transmit signals (e.g. data) e.g. New Radio (NR), Wi-Fi, Long Term Evolution (LTE), LTE-Advanced, Wideband Code Division Multiple Access (WCDMA), Global System for Mobile communications/enhanced Data rate for GSM Evolution (GSM/EDGE), Worldwide Interoperability for Microwave Access (WiMax), or Ultra Mobile Broadband (UMB), just to mention a few possible implementations.

Any appropriate steps, methods, features, functions, or benefits disclosed herein may be performed through one or more functional units or modules of one or more virtual apparatuses. Each virtual apparatus may comprise a number of these functional units. These functional units may be implemented via processing circuitry, which may include one or more microprocessor or microcontrollers, as well as other digital hardware, which may include digital signal processors (DSPs), special-purpose digital logic, and the like. The processing circuitry may be configured to execute program code stored in memory, which may include one or several types of memory such as read-only memory (ROM), random-access memory (RAM), cache memory, flash memory devices, optical storage devices, etc. Program code stored in memory includes program instructions for executing one or more telecommunications and/or data communications protocols as well as instructions for carrying out one or more of the techniques described herein. In some implementations, the processing circuitry may be used to cause the respective functional unit to perform corresponding functions according one or more embodiments of the present disclosure.

As will be readily understood by those familiar with communications design, that functions means or modules may be implemented using digital logic and/or one or more microcontrollers, microprocessors, or other digital hardware. In some embodiments, several or all of the various functions may be implemented together, such as in a single application-specific integrated circuit (ASIC), or in two or more separate devices with appropriate hardware and/or software interfaces between them. Several of the functions may be implemented on a processor shared with other functional components of a radio network node or UE, for example.

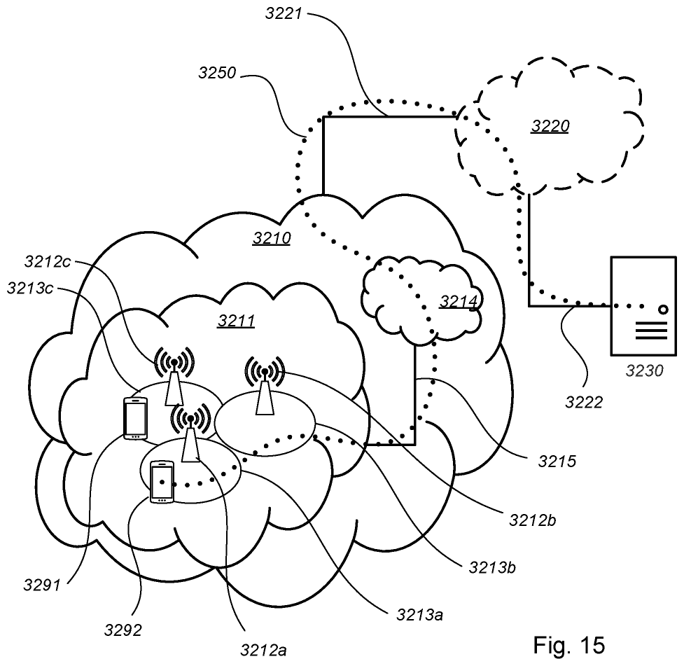

With reference to FIG. 15, in accordance with an embodiment, a communication system includes a telecommunication network 3210, such as a 3GPP-type cellular network, which comprises an access network 3211, such as a radio access network, and a core network 3214. The access network 3211 comprises a plurality of base stations 3212a, 3212b, 3212c, such as NBs, eNBs, gNBs or other types of wireless access points being examples of the network node 12 herein, each defining a corresponding coverage area 3213a, 3213b, 3213c. Each base station 3212a, 3212b, 3212c is connectable to the core network 3214 over a wired or wireless connection 3215. A first user equipment (UE) 3291, being an example of the monitoring device 10, located in coverage area 3213c is configured to wirelessly connect to, or be paged by, the corresponding base station 3212c. A second UE 3292 in coverage area 3213a is wirelessly connectable to the corresponding base station 3212a. While a plurality of UEs 3291, 3292 are illustrated in this example, the disclosed embodiments are equally applicable to a situation where a sole UE is in the coverage area or where a sole UE is connecting to the corresponding base station 3212.

The telecommunication network 3210 is itself connected to a host computer 3230, which may be embodied in the hardware and/or software of a standalone server, a cloud-implemented server, a distributed server or as processing resources in a server farm. The host computer 3230 may be under the ownership or control of a service provider, or may be operated by the service provider or on behalf of the service provider. The connections 3221, 3222 between the telecommunication network 3210 and the host computer 3230 may extend directly from the core network 3214 to the host computer 3230 or may go via an optional intermediate network 3220. The intermediate network 3220 may be one of, or a combination of more than one of, a public, private or hosted network; the intermediate network 3220, if any, may be a backbone network or the Internet; in particular, the intermediate network 3220 may comprise two or more sub-networks (not shown).

The communication system of FIG. 15 as a whole enables connectivity between one of the connected UEs 3291, 3292 and the host computer 3230. The connectivity may be described as an over-the-top (OTT) connection 3250. The host computer 3230 and the connected UEs 3291, 3292 are configured to communicate data and/or signalling via the OTT connection 3250, using the access network 3211, the core network 3214, any intermediate network 3220 and possible further infrastructure (not shown) as intermediaries. The OTT connection 3250 may be transparent in the sense that the participating communication devices through which the OTT connection 3250 passes are unaware of routing of uplink and downlink communications. For example, a base station 3212 may not or need not be informed about the past routing of an incoming downlink communication with data originating from a host computer 3230 to be forwarded (e.g., handed over) to a connected UE 3291. Similarly, the base station 3212 need not be aware of the future routing of an outgoing uplink communication originating from the UE 3291 towards the host computer 3230.

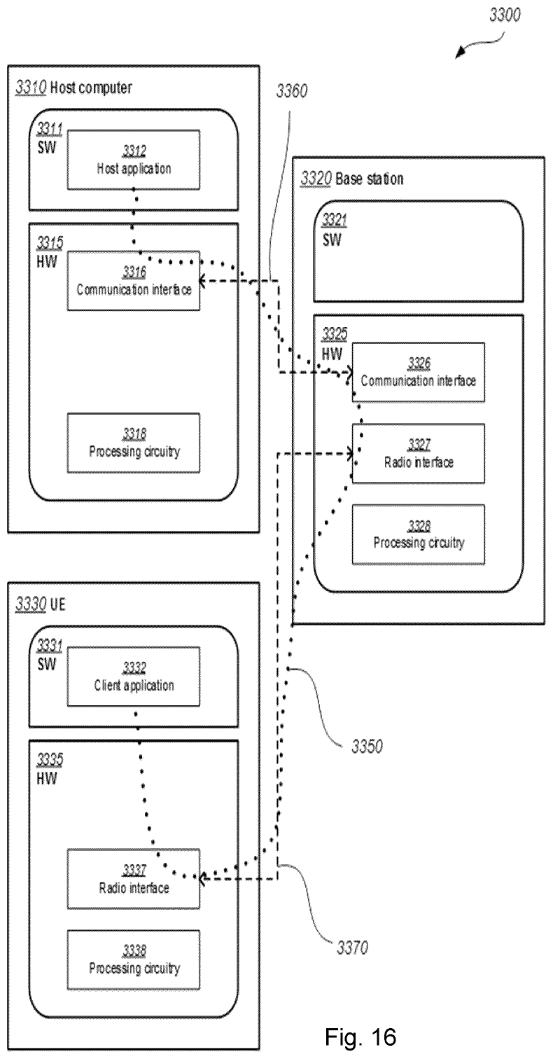

Example implementations, in accordance with an embodiment, of the UE, base station and host computer discussed in the preceding paragraphs will now be described with reference to FIG. 16. In a communication system 3300, a host computer 3310 comprises hardware 3315 including a communication interface 3316 configured to set up and maintain a wired or wireless connection with an interface of a different communication device of the communication system 3300. The host computer 3310 further comprises processing circuitry 3318, which may have storage and/or processing capabilities. In particular, the processing circuitry 3318 may comprise one or more programmable processors, application-specific integrated circuits, field programmable gate arrays or combinations of these (not shown) adapted to execute instructions. The host computer 3310 further comprises software 3311, which is stored in or accessible by the host computer 3310 and executable by the processing circuitry 3318. The software 3311 includes a host application 3312. The host application 3312 may be operable to provide a service to a remote user, such as a UE 3330 connecting via an OTT connection 3350 terminating at the UE 3330 and the host computer 3310. In providing the service to the remote user, the host application 3312 may provide user data which is transmitted using the OTT connection 3350.

The communication system 3300 further includes a base station 3320 provided in a telecommunication system and comprising hardware 3325 enabling it to communicate with the host computer 3310 and with the UE 3330. The hardware 3325 may include a communication interface 3326 for setting up and maintaining a wired or wireless connection with an interface of a different communication device of the communication system 3300, as well as a radio interface 3327 for setting up and maintaining at least a wireless connection 3370 with a UE 3330 located in a coverage area (not shown in FIG. 16) served by the base station 3320. The communication interface 3326 may be configured to facilitate a connection 3360 to the host computer 3310. The connection 3360 may be direct or it may pass through a core network (not shown in FIG. 16) of the telecommunication system and/or through one or more intermediate networks outside the telecommunication system. In the embodiment shown, the hardware 3325 of the base station 3320 further includes processing circuitry 3328, which may comprise one or more programmable processors, application-specific integrated circuits, field programmable gate arrays or combinations of these (not shown) adapted to execute instructions. The base station 3320 further has software 3321 stored internally or accessible via an external connection.

The communication system 3300 further includes the UE 3330 already referred to. Its hardware 3335 may include a radio interface 3337 configured to set up and maintain a wireless connection 3370 with a base station serving a coverage area in which the UE 3330 is currently located. The hardware 3335 of the UE 3330 further includes processing circuitry 3338, which may comprise one or more programmable processors, application-specific integrated circuits, field programmable gate arrays or combinations of these (not shown) adapted to execute instructions. The UE 3330 further comprises software 3331, which is stored in or accessible by the UE 3330 and executable by the processing circuitry 3338. The software 3331 includes a client application 3332. The client application 3332 may be operable to provide a service to a human or non-human user via the UE 3330, with the support of the host computer 3310. In the host computer 3310, an executing host application 3312 may communicate with the executing client application 3332 via the OTT connection 3350 terminating at the UE 3330 and the host computer 3310. In providing the service to the user, the client application 3332 may receive request data from the host application 3312 and provide user data in response to the request data. The OTT connection 3350 may transfer both the request data and the user data. The client application 3332 may interact with the user to generate the user data that it provides.

It is noted that the host computer 3310, base station 3320 and UE 3330 illustrated in FIG. 16 may be identical to the host computer 3230, one of the base stations 3212a, 3212b, 3212c and one of the UEs 3291, 3292 of FIG. 15, respectively. This is to say, the inner workings of these entities may be as shown in FIG. 16 and independently, the surrounding network topology may be that of FIG. 15.

In FIG. 15, the OTT connection 3350 has been drawn abstractly to illustrate the communication between the host computer 3310 and the user equipment 3330 via the base station 3320, without explicit reference to any intermediary devices and the precise routing of messages via these devices. Network infrastructure may determine the routing, which it may be configured to hide from the UE 3330 or from the service provider operating the host computer 3310, or both. While the OTT connection 3350 is active, the network infrastructure may further take decisions by which it dynamically changes the routing (e.g., on the basis of load balancing consideration or reconfiguration of the network).

The wireless connection 3370 between the UE 3330 and the base station 3320 is in accordance with the teachings of the embodiments described throughout this disclosure. One or more of the various embodiments improve the performance of OTT services provided to the UE 3330 using the OTT connection 3350, in which the wireless connection 3370 forms the last segment. More precisely, the teachings of these embodiments may achieve an efficient and secure gathering of sensor data and thereby provide benefits such as reduced waiting time, improved battery time, and better responsiveness.

A measurement procedure may be provided for the purpose of monitoring data rate, latency and other factors on which the one or more embodiments improve. There may further be an optional network functionality for reconfiguring the OTT connection 3350 between the host computer 3310 and UE 3330, in response to variations in the measurement results. The measurement procedure and/or the network functionality for reconfiguring the OTT connection 3350 may be implemented in the software 3311 of the host computer 3310 or in the software 3331 of the UE 3330, or both. In embodiments, sensors (not shown) may be deployed in or in association with communication devices through which the OTT connection 3350 passes; the sensors may participate in the measurement procedure by supplying values of the monitored quantities exemplified above, or supplying values of other physical quantities from which software 3311, 3331 may compute or estimate the monitored quantities. The reconfiguring of the OTT connection 3350 may include message format, retransmission settings, preferred routing etc.; the reconfiguring need not affect the base station 3320, and it may be unknown or imperceptible to the base station 3320. Such procedures and functionalities may be known and practiced in the art. In certain embodiments, measurements may involve proprietary UE signalling facilitating the host computer's 3310 measurements of throughput, propagation times, latency and the like. The measurements may be implemented in that the software 3311, 3331 causes messages to be transmitted, in particular empty or ‘dummy’ messages, using the OTT connection 3350 while it monitors propagation times, errors etc.

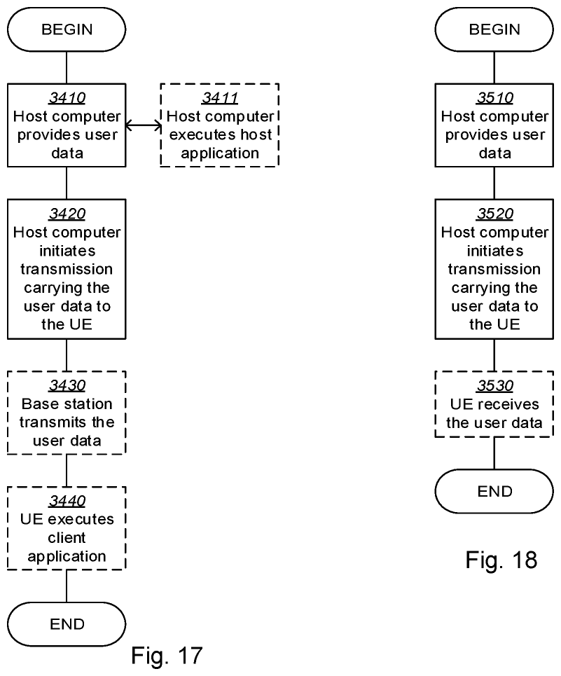

FIG. 17 is a flowchart illustrating a method implemented in a communication system, in accordance with one embodiment. The communication system includes a host computer, a base station and a UE which may be those described with reference to FIGS. 15 and 16. For simplicity of the present disclosure, only drawing references to FIG. 17 will be included in this section. In a first step 3410 of the method, the host computer provides user data. In an optional substep 3411 of the first step 3410, the host computer provides the user data by executing a host application. In a second step 3420, the host computer initiates a transmission carrying the user data to the UE. In an optional third step 3430, the base station transmits to the UE the user data which was carried in the transmission that the host computer initiated, in accordance with the teachings of the embodiments described throughout this disclosure. In an optional fourth step 3440, the UE executes a client application associated with the host application executed by the host computer.

FIG. 18 is a flowchart illustrating a method implemented in a communication system, in accordance with one embodiment. The communication system includes a host computer, a base station and a UE which may be those described with reference to FIGS. 15 and 16. For simplicity of the present disclosure, only drawing references to FIG. 18 will be included in this section. In a first step 3510 of the method, the host computer provides user data. In an optional substep (not shown) the host computer provides the user data by executing a host application. In a second step 3520, the host computer initiates a transmission carrying the user data to the UE. The transmission may pass via the base station, in accordance with the teachings of the embodiments described throughout this disclosure. In an optional third step 3530, the UE receives the user data carried in the transmission.

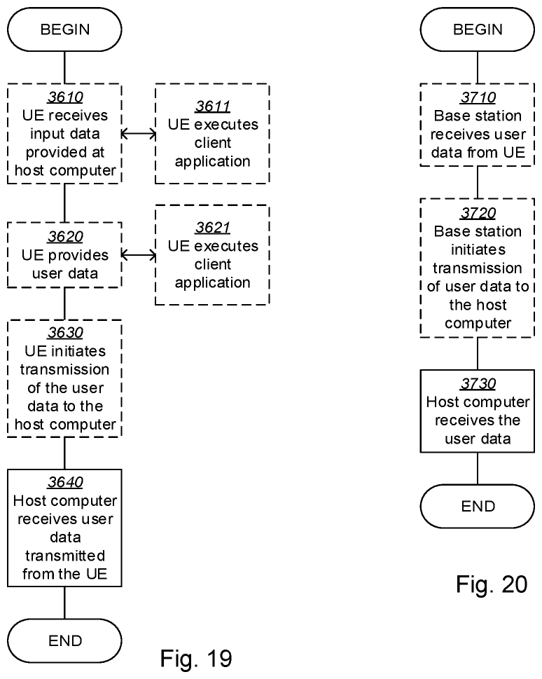

FIG. 19 is a flowchart illustrating a method implemented in a communication system, in accordance with one embodiment. The communication system includes a host computer, a base station and a UE which may be those described with reference to FIGS. 15 and 16. For simplicity of the present disclosure, only drawing references to FIG. 19 will be included in this section. In an optional first step 3610 of the method, the UE receives input data provided by the host computer. Additionally or alternatively, in an optional second step 3620, the UE provides user data. In an optional substep 3621 of the second step 3620, the UE provides the user data by executing a client application. In a further optional substep 3611 of the first step 3610, the UE executes a client application which provides the user data in reaction to the received input data provided by the host computer. In providing the user data, the executed client application may further consider user input received from the user. Regardless of the specific manner in which the user data was provided, the UE initiates, in an optional third substep 3630, transmission of the user data to the host computer. In a fourth step 3640 of the method, the host computer receives the user data transmitted from the UE, in accordance with the teachings of the embodiments described throughout this disclosure.

FIG. 20 is a flowchart illustrating a method implemented in a communication system, in accordance with one embodiment. The communication system includes a host computer, a base station and a UE which may be those described with reference to FIGS. 15 and 16. For simplicity of the present disclosure, only drawing references to FIG. 20 will be included in this section. In an optional first step 3710 of the method, in accordance with the teachings of the embodiments described throughout this disclosure, the base station receives user data from the UE. In an optional second step 3720, the base station initiates transmission of the received user data to the host computer. In a third step 3730, the host computer receives the user data carried in the transmission initiated by the base station.

It will be appreciated that the foregoing description and the accompanying drawings represent non-limiting examples of the methods and apparatus taught herein. As such, the apparatus and techniques taught herein are not limited by the foregoing description and accompanying drawings. Instead, the embodiments herein are limited only by the following claims and their legal equivalents.

1. A method performed by a monitoring device for handling brain activity data of a user, the method comprising:
obtaining sensor data from a plurality of sensors, wherein the sensor data indicates a brain activity of one or more regions in a brain of the user;
constructing a similarity module of brain activities based on an activity configuration defining one or more sensors out of the plurality of sensors to gather sensor data from and obtained sensor data from the one or more sensors, wherein the similarity module comprises one or more groups of sensor data related to one another;
performing an interaction analysis within and/or between the one or more groups in the similarity module by creating a network of interactions within and/or between the one or more groups;
creating an aggregated activity graph based on the constructed similarity module, the performed interaction analysis, and a configured aggregation level of sensor data; and
providing the aggregated activity graph to a network node.

2. The method according to claim 1, further comprising
obtaining the activity configuration and an indication of the configured aggregation level of sensor data from within the monitoring device, or from the network node.

3. The method according to claim 1, wherein the one or more sensors are related to one or more regions of the brain to monitor, and the similarity module comprises a similarity matrix constructed from sensor data of the one or more regions, and the network of interactions is computed by performing community analysis on the similarity matrix.

4. The method according to claim 1, wherein creating the aggregated activity graph comprises comparing the created network of interactions with a reference network of interactions and forming the aggregated activity graph based on said comparison.

5. The method according to claim 4, wherein a compared network of interactions is obtained from the comparison, and wherein creating the aggregated activity graph further comprises constructing the aggregated activity graph by aggregating the compared network of interactions into the aggregated activity graph.

6. (canceled)

7. A method performed by a network node for handling brain activity data of a user, the method comprising:
receiving from a monitoring device related to the user, an aggregated activity graph, wherein the aggregated activity graph is based on a similarity module, an interaction analysis performed at the monitoring device, and a configured aggregation level of sensor data, wherein the sensor data indicates a brain activity of one or more regions in a brain of the user; and
initiating an action related to an experience of the user based on the received aggregated activity graph.

8. The method according to claim 7, further comprising
providing to the monitoring device, an activity configuration defining one or more sensors out of a plurality of sensors to gather sensor data from, and an indication of the configured aggregation level of sensor data.

9. The method according to claim 8, further comprising
updating the activity configuration based on one or more received aggregated activity graphs.

10. The method according to claim 9, wherein updating the activity configuration comprises:
receiving a further aggregated activity graph from another monitoring device;
comparing the received aggregated activity graph and the further aggregated activity graph to group aggregated activity graphs of users;
computing a reference network of interactions for the users of the grouped aggregated activity graphs; and
sending the computed reference network of interactions to monitoring devices of the users.

11. The method according to claim 7, wherein the action initiated comprises one or more of the following:
selecting content to provide to the user based on the received aggregated activity graph;
recommending a product or a service to the user based on the received aggregated activity graph;
recommending an action for a third party to perform based on the received aggregated activity graph;

12.-13. (canceled)

14. A monitoring device for handling brain activity data of a user, wherein the monitoring device is configured to:
obtain sensor data from a plurality of sensors, wherein the sensor data indicates a brain activity of one or more regions in a brain of the user;
construct a similarity module of brain activities based on an activity configuration defining one or more sensors out of the plurality of sensors to gather sensor data from and obtained sensor data from the one or more sensors, wherein the similarity module comprises one or more groups of sensor data related to one another;
perform an interaction analysis within and/or between the one or more groups in the similarity module by creating a network of interactions within and/or between the one or more groups;
create an aggregated activity graph based on the constructed similarity module, the performed interaction analysis, and a configured aggregation level of sensor data; and
provide the aggregated activity graph to a network node.

15. The monitoring device according to claim 14, wherein the monitoring device is configured to
obtain the activity configuration and an indication of the configured aggregation level of sensor data from within the monitoring device, or from the network node.

16. The monitoring device according to claim 14, wherein the one or more sensors are related to one or more regions of the brain to monitor, and the similarity module comprises a similarity matrix constructed from sensor data of the one or more regions, and the monitoring device is configured to compute the network of interactions by performing community analysis on the similarity matrix.

17. The monitoring device according to claim 14, wherein the monitoring device is configured to create the aggregated activity graph by comparing the created network of interactions with a reference network of interactions and forming the aggregated activity graph based on said comparison.

18. The monitoring device according to claim 17, wherein a compared network of interactions is obtained from the comparison, and wherein the monitoring device is configured to create the aggregated activity graph by aggregating the compared network of interactions into the aggregated activity graph.

19. (canceled)

20. A network node for handling brain activity data of a user, wherein the network node is configured to:
receive from a monitoring device related to the user, an aggregated activity graph, wherein the aggregated activity graph is based on a similarity module, an interaction analysis performed at the monitoring device, and a configured aggregation level of sensor data, wherein the sensor data indicates a brain activity of one or more regions in a brain of the user; and
initiate an action related to an experience of the user based on the received aggregated activity graph.

21. The network node according to claim 20, wherein the network node is configured to
provide to the monitoring device, an activity configuration defining one or more sensors out of a plurality of sensors to gather sensor data from, and an indication of the configured aggregation level of sensor data.

22. The network node according to claim 21, wherein the network node is further configured to
update the activity configuration based on one or more received aggregated activity graphs.

23. The network node according to claim 20, wherein the network node is further configured to
receive a further aggregated activity graph from another monitoring device;
compare the received aggregated activity graph and the further aggregated activity graph to group aggregated activity graphs of users;
compute a reference network of interactions for the users of the grouped aggregated activity graphs; and
send the computed reference network of interactions to monitoring devices of the users.

24. The network node according to claim 20, wherein the action initiated comprises one or more of the following:
selecting content to provide to the user based on the received aggregated activity graph;
recommending a product or a service to the user based on the received aggregated activity graph;
recommending an action for a third party to perform based on the received aggregated activity graph.
